(19) United States
(12) Patent Application Publication
(10) Pub. No.: US 2001/0039019 A1
(43) Pub. Date: Nov. 8, 2001

(54) METHOD FOR THE IN VITRO DIAGNOSIS OF COELIAC DISEASE BY SECONDARY ANTIBODIES

(76) Inventor: ANTONIO PICARELLI, ROME (IT)

Correspondence Address: STEVENS DAVIS MILLER & MOSHER, SUITE 1615, 1615 L STREET NW, WASHINGTON, DC, 20036

(21) Appl. No.: 09/215,142

(22) Filed: Dec. 18, 1998

(51) Int. Cl. G01N 33/53; G01N 33/567; G01N 33/537; G01N 33/543

(52) U.S. Cl. 435/7.1; 435/7.2; 435/7.95; 435/7.92

(57) ABSTRACT

The diagnosis in vitro of the coeliac disease is carried out by using secondary antibodies, an antibody of IgA class and an antibody of IgG1 class, as means for detecting the presence of antiendomysial antibodies both in blood serum and culture media of the enteral mucosa.

### FIELD OF THE INVENTION

The present invention relates to the diagnostics and more particularly the diagnosis in vitro of the coeliac disease by means of two secondary antibodies. Specifically the invention makes use of antihuman antibodies of IgG1 class in addition to antihuman antibodies of IgA class as means for detecting the presence of antiendomysial antibodies both in blood serum and culture media of the enteral mucosa.

### BACKGROUND OF THE INVENTION

The coeliac disease is a common gastroenterologic pathology, the diagnosis of which is conventionally made by a hystologic test of the enteral mucosa. The diagnosis is based on the detection of villar atrophy in the duodenal and/or jejunal mucosa which subsides after exclusion of gluten from the diet.

It has been known for some time that the test of antiendomysial antibodies is positive in most subjects suffering from coeliac disease.

Recent works have demonstrated that the presence of antiendomysial antibodies in the serum of subjects free from villar atrophy is due to a condition of false negativeness of the conventional hystologic test.

It has been further proved that an evidence of the coeliac disease is the production of antiendomysial antibodies by the enteral mucosa in suitable culture media.

The literature also discloses false negative tests for antiendomysial antibodies in case of total or subtotal villar atrophy: statistic data state that the specificity of the antiendomysial antibodies is 100% while their sensitivity is lower, i.e. near 95%. Antiendomysial antibodies are detected by a secondary antibody of IgA class today; in fact, antiendomysial antibodies of IgG class are not present in subjects suffering from coeliac disease according to the present knowledge.

As a result of an exhaustive research conducted by the Applicant on a number of subjects, it has been surprisingly found that many people are positive to the test of antiendomysial antibodies (EMA) of IgG class.

It has also been proved that such antibodies belong to IgG1 subclass.

It should be noted that the positiveness of the antiendomysial antibodies of G1 class is not exclusively present in subjects with total deficit of IgA. Such a condition is found only in 14% of the patient.

The applicant has then found that about 20% EMA-positive coeliac patients produce simultaneously antibodies of the two classes A and G1.

Examinations of the enteral mucosa have shown that also mucosa of the EMA G1-positive patients produces the same antibodies.

A statistic wide-ranging indagation has proved that normal subjects do not produce antiendomysial antibodies either of class A or of class G and relative subclasses (1 4) either in blood serum or culture liquids of the enteral biopsies.

### SUMMARY OF THE INVENTION

The inventor started from the above results and disclosed a method for the diagnosis in vitro of the coeliac disease which makes use of antihuman antibodies of IgG1 class in addition to antihuman antibodies of IgA class as means for detecting the presence of antiendomysial antibodies both in blood serum and culture media of the enteral mucosa.

Therefore, a right diagnosis according to the invention needs the separate use of two secondary antibodies, namely:

a) antihuman IgA and

b) antihuman IgG1.

### DETAILED DESCRIPTION OF THE PREFERRED EMBODIMENT

According to a preferred embodiment the method of the present invention includes the following steps:

a) incubating with the serum or the culture liquid of the enteral biopsy of the patient on sections of monkey's oesophagus, umbilical cord or other suitable substrate in two separate tanks for half a hour; in case serum is used, the same is diluted in PBS (phosphate buffer solution) in a ratio of 1:2 to 1:8;

b) washing;

c) incubating with secondary antibody IgA suitably marked in one of the two tanks still for half a hour;

d) incubating with secondary antibody IgG1 suitably marked in the other tank still for half a hour; said secondary antibody being diluted in PBS in a ratio of 1:50 to 1:140, preferably 1:100;

e) microscope watching with development of specific markers, e.g. fluorescein (FITC).

A kit for the diagnosis in vitro of the coeliac disease according to the invention includes at least one secondary antibody of IgA class and one secondary antibody of IgG1 class.

A number of 200 sera from patients aged between 2 and 72 years were tested by the above described method. The sensitivity of the system is 100% if compared with the culture data.

The specificity of the system is 100% if compared with the culture data.

The test was carried out on EMA-A negative patients having, however, clinic, instrumental laboratory evidences of coeliac disease.

A preferred embodiment of the invention has been described, however, it is self evident that changes and modifications can be made by those skilled in the art without departing from the scope of the present industrial invention as claimed in the appended claims.

1. Use of an antihuman antibody of IgG1 class as means for detecting the presence of antiendomysial antibodies both in blood serum and culture media of the enteral mucosa.

2. A method for the diagnosis in vitro of the coeliac disease, characterized in that it makes use of antihuman antibodies of IgG1 class in addition to antihuman antibodies of IgA class as means for detecting the presence of antiendomysial antibodies both in blood serum and culture media of the enteral mucosa.

3. The method of claim 2, characterized in that there are provided the following steps:
a) incubating with the serum or the culture liquid of the enteral biopsy of the patient on sections of monkey's oesophagus, umbilical cord or other suitable substrate in two separate tanks for half a hour;
b) washing;
c) incubating with secondary antibody IgA suitably marked in one of the two tanks still for half a hour;
d) incubating with secondary antibody IgG1 suitably marked in the other tank still for half a hour;
e) microscope watching with development of specific markers.

4. The method of claim 3, characterized in that, in case serum is used, the same is diluted in PBS (phosphate buffer solution) in a ratio of 1:2 to 1:8.

5. The method of claim 3, characterized in that the serum is diluted in PBS in a ratio of 1:4.

6. The method of claim 3, characterized in that the enteral biopsy culture liquid is not diluted.

7. The method of claim 3, characterized in that secondary antibody IgG1 is diluted in PBS in a ratio of 1:50 to 1:140.

8. The method of claim 3, characterized in that secondary antibody IgG1 is diluted in PBS in a ratio of 1:100.

9. The method of claim 3, characterized in that the incubation time of step a) is in the order of half a hour.

10. The method of claim 3, characterized in that the incubation time of steps c) and d) is in the order of half a hour.

11. The method of claim 3, characterized in that the watching of step b) is carried out in PBS.

12. A kit for the diagnosis of the coeliac disease, characterized in that at least one secondary antibody of IgG1 class is provided.

13. A kit for the diagnosis of the coeliac disease, characterized in that at least one secondary antibody of IgA class and one secondary antibody of IgG1 class are provided as means for detecting the presence of antiendomysial antibodies both in blood serum and culture media of the enteral mucosa.

14. A method for the diagnosis in vitro of the coeliac disease as substantially disclosed in the description and claimed in the present claims.
